(19) United States
(12) Patent Application Publication
(10) Pub. No.: US 2018/0372728 A1
(43) Pub. Date: Dec. 27, 2018

(54) BIOMEDICAL DETECTION DEVICES USING MAGNETIC BIOSENSORS

(71) Applicant: PINGCHIECH WU, Taipei City (TW)

(72) Inventor: PINGCHIECH WU, Taipei City (TW)

(21) Appl. No.: 15/632,942

(22) Filed: Jun. 26, 2017

(51) Int. Cl. G01N 33/53; G01N 35/00; G01R 33/02

(52) U.S. Cl. CPC G01N 33/53; G01N 27/72; G01R 33/0213; G01N 35/0098

(57) ABSTRACT

A biomedical detection device includes a sample cell, an electromagnetic generating unit, a light source and a light detection unit. The sample cell can be filled with a liquid sample containing a detection object and a magnetic biosensor capable of combining the detection object to form a magnetic cluster. The electromagnetic generating unit is disposed at opposite two sides of the sample cell, and capable of generating an oscillating magnetic field of single frequency to drive motions of the magnetic biosensor and the magnetic cluster. The light source is disposed at a side of the sample cell, and capable of emitting light rays to pass through the liquid sample. The light detection unit is disposed at another side of the sample cell oppositely to the light source, and capable of outputting electrical signals based on a change of the light rays.

### BACKGROUND OF THE INVENTION

### 1. Field of the Invention

The present application relates to a detection device, especially to a biomedical detection device using magnetic biosensors.

### 2. The Prior Arts

Due conventional labeling techniques in immunoassay, such as the sandwich method, includes multiple bonding steps and washing steps, the processes thereof need several hours to be completed, the processes of some detection methods further need to add fluorescent or radioactive material to perform the labeling reactions. In addition to the complicated operation procedures, the detection results of conventional immunoassay easily have errors caused by human factors, and the detection sensitivities thereof are still insufficient. For improving the drawbacks of the conventional labeling immunoassay, a magneto-optic biosensor is being developed by using magnetic biosensors as a label. In addition to high sensitivity, the magneto-optic biosensor further has an advantage that can allow the magnetic biosensor move in a liquid by using an external magnetic field, so as to increase the probability of combining the magnetic biosensor and the detection object, thereby to reduce the detection time.

U.S. Pat. No. 7,639,359 discloses a method that detects dynamic trajectory of magnetic sensors in a liquid by utilizing FARADAY magneto-optic effect, the method includes following steps: combining the magnetic biosensors with the detection object to form magnetic clusters; driving the magnetic clusters move with a magnetic field of different frequencies; transmitting polarized light rays through the liquid containing the magnetic clusters; detecting the frequencies of the polarized light rays and a motion curve of the magnetic clusters by using a light detection unit and a lock-in amplifier; and calculating an amount of the detection object with the motion curve. Because of using the polarized light rays to detect the motion curve of the magnetic clusters, the method disclosed by U.S. Pat. No. 7,639,359 needs to use polarized lenses to filter unneeded light rays, a structure of the detection device thereof is complicated, and a detection cost thereof is high.

Patent publication WO 2014/206584 discloses a method that detects dynamic trajectory of magnetic sensors in a liquid, the method, same as the method disclosed by U.S. Pat. No. 7,639,359, drives the magnetic clusters move with different frequencies, and calculates a motion curve of the magnetic clusters under the different frequencies by using a lock-in amplifier; a detection device thereof does not use polarized lenses (quarter-wave plates); therefore, a structure thereof is simpler, and a detection cost is lower. The method disclosed by Patent publication WO 2014/206584 can improve the drawback of using the polarized lenses disclosed by U.S. Pat. No. 7,639,359, to detect an amount of the detection object at a lower cost; however, the method disclosed thereby needs to scan the different frequencies of the magnetic field to detect the motion curve, so that a detection time is longer.

Therefore, there is still a need for a detection device that can accurately detect the amount of objects to be detected and magnetic biosensors in a short time.

### SUMMARY OF THE INVENTION

In order to meet the need, the present application provides a biomedical detection device, which includes a sample cell, an electromagnetic generating unit, a light source and a light detection unit. The sample cell can be filled with a liquid sample containing a detection object and a magnetic biosensor capable of combining the detection object to form a magnetic cluster. The electromagnetic generating unit is disposed at opposite two sides of the sample cell, and capable of generating an oscillating magnetic field of single frequency to drive motions of the magnetic biosensor and the magnetic cluster. The light source is disposed at a side of the sample cell, and capable of emitting light rays to pass through the liquid sample. The light detection unit is disposed at another side of the sample cell oppositely to the light source, and capable of outputting electrical signals based on a change of the light rays caused by the motions of the magnetic biosensor and the magnetic cluster when the light rays pass through the liquid sample.

The biomedical detection device according to the present application performs the immunoassay by using the magnetic nanoparticles as a label includes steps as follows. Firstly, a biosensor having a combining uniqueness to the detection object is selected, and the selected biosensor is coated on a surface of a magnetic nanoparticle to form a magnetic biosensor, while the magnetic biosensor and the detection object are mixed in a liquid, a magnetic cluster can be formed by combining the magnetic biosensor and the detection object. Secondly, the liquid containing the magnetic biosensor and the detection object is filled in a sample cell, and an oscillating magnetic field of single frequency is generated by an electromagnetic generating unit which is disposed at opposite two sides of the sample cell, the oscillating magnetic field drives motions of the magnetic biosensor and the magnetic cluster. Then, the light source which is disposed at a side of the sample cell emits light rays to pass through the liquid sample in the sample cell, the magnetic biosensor and the magnetic cluster are rotated by the oscillating magnetic field, so intensities of the light rays passing through the sample cell change. The light detection unit receives the light rays that passed through the sample cell to output electrical signals of a frequency being two times of the magnetic field frequency, and a change of the electrical signal is proportional to the change of the light ray intensities. Finally, an amount of the detection object can be calculated, by comparing a difference of the change of electrical signals before and after that the magnetic biosensor and the detection object are combined.

In order to accurately measure the change of the electrical signals of the light detection unit, the electrical signals output by light detection unit are input a lock-in amplifier, which calculates an in-phase component and a quadrature component based on the electrical signals output by the light detection unit, the change of the electrical signals output by the light detection unit can be accurately obtain by the calculation of the in-phase component and the quadrature component.

In the biomedical detection device of the present application, the magnetic biosensor can combine the detection object to form the magnetic cluster, the electromagnetic generating unit can generate an oscillating magnetic field of the single frequency to drive motions of the magnetic biosensor and the magnetic cluster, the light detection unit can output electrical signals based on an change of the light rays caused by motions of the magnetic biosensor and the magnetic cluster, when the light rays pass through the liquid sample, and the amount of the detection object in the liquid sample can rapidly and accurately be obtained through calculation of the change of the electrical signals output by the light detection unit.

### BRIEF DESCRIPTION OF THE DRAWINGS

The present application can be understood in more detail by reading the subsequent detailed description in conjunction with the examples and references made to the accompanying drawings, in which:

FIG. 1 is a side view schematically illustrating a biomedical detection device of the present application;

FIG. 2 is a side view schematically illustrating a state in the sample cell that the magnetic biosensor not yet combines the detection object;

FIG. 3 is an oscillogram illustrating the electrical signals output by the light detection unit corresponding to the magnetic biosensor at different positions;

FIG. 4 is a side view schematically illustrating a state in the sample cell that the magnetic biosensor combines the detection object to form a magnetic cluster;

FIG. 5 is an oscillogram illustrating the electrical signals output by the light detection unit corresponding to the magnetic cluster at different positions;

FIG. 6 is an oscillogram illustrating the electrical signals which are input the lock-in amplifier; and

FIG. 7 is a scatter diagram illustrating the detection data of the electrical signal change to the detection objects of respective concentrations according to an embodiment of the present application.

### DETAILED DESCRIPTION OF THE PREFERRED EMBODIMENTS

The present application may be embodied in various forms and the details of the preferred embodiments of the present application will be described in the subsequent content with reference to the accompanying drawings. The drawings (not to scale) show and depict only the preferred embodiments of the invention and shall not be considered as limitations to the scope of the present application. Modifications of the shape of the present application shall be considered to be within the spirit of the present application.

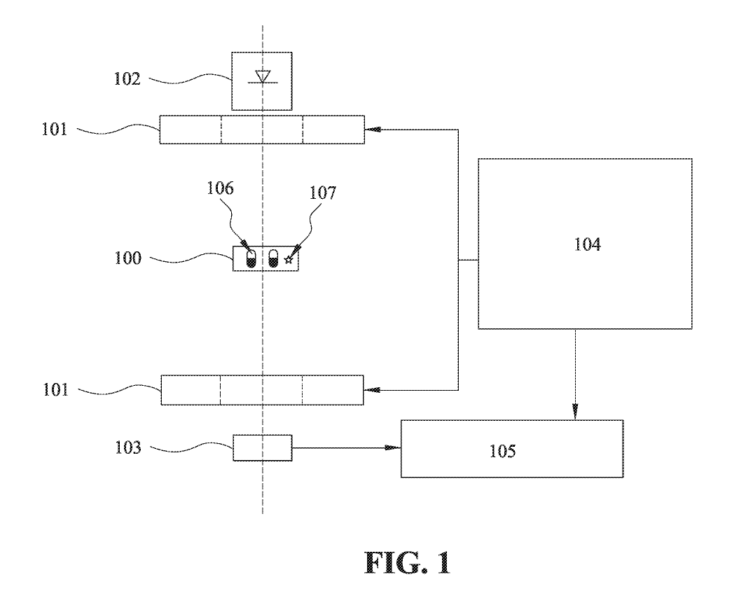

FIG. 1 is a side view schematically illustrating a biomedical detection device of the present application. As shown in FIG. 1, a biomedical detection device includes a sample cell 100, an electromagnetic generating unit 101, a light source 102, a light detection unit 103, a digital signal processor (DSP) 104 and a lock-in amplifier 105. The electromagnetic generating unit 101 is disposed at opposite both sides on a vertical direction of a sample cell 100; the light source 102 and the light detection unit 103 are respectively disposed at upper and lower sides of the sample cell 100, and outside the electromagnetic generating unit 101. The digital signal processor 104 is used for controlling the electromagnetic generating unit 101. The lock-in amplifier 105 connects to the light detection unit 103 and the digital signal processor 104.

The sample cell 100 has micro-fluidic channels formed on a horizontal direction, and a magnetic biosensor 106 can combine a detection object 107 contained in a liquid sample through the micro-fluidic channels to form a magnetic cluster. The DSP 104 can output an oscillating signal to allow the electromagnetic generating unit 101 generates an oscillating magnetic field, and magnetic force lines thereof penetrate the sample cell 100 in a substantially vertical manner. Light rays emitted by a light source 102 pass through the liquid sample in the sample cell 100 from top to bottom, and are received by a light detection unit 103.

In embodiments of the present application, a concentration of the magnetic biosensor 106 in the liquid sample is 0.1-2000 μg/mL. The electromagnetic generating unit 101 is a pair of electromagnets, the single frequency of the oscillating magnetic field is 1-500 Hz, and an intensity of the oscillating magnetic field is 1-100 mT. A wavelength of the light rays emitted by the light source 102 is under 650 nm, and the light source 102 includes a polarized laser. The light detection unit 103 includes photodiodes, and electrical signals output by the light detection unit 103 are voltage signals.

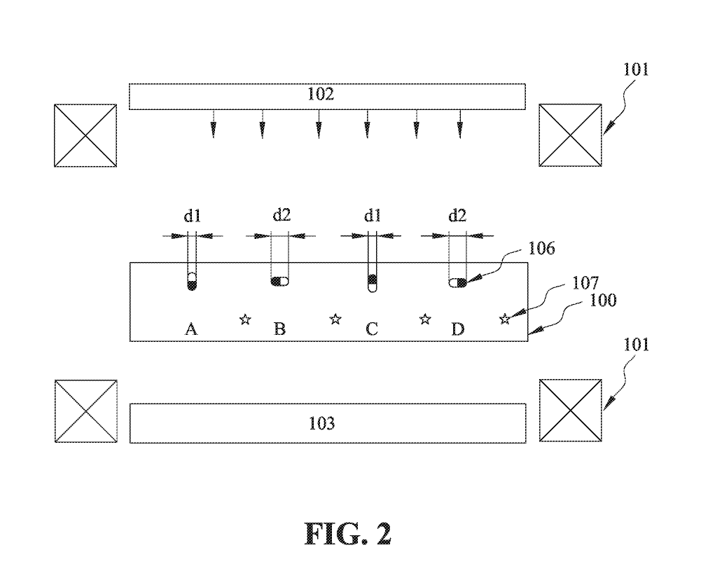

FIG. 2 is a side view schematically illustrating a state in the sample cell that the magnetic biosensor not yet combines the detection object. As shown in FIG. 2, before the magnetic biosensor 106 combines the detection object 107 to form a magnetic cluster, the oscillating magnetic field generated by the electromagnetic generating unit 101 drives the magnetic biosensor 106 rotating along a direction of positions A, B, C and D. When the light rays emitted by the light source 102 pass through the liquid sample, a change of the light rays that are affected by the rotation of the magnetic biosensor 106 occurs. The light detection unit 103 detects and measures the change of the light rays, and outputs the electrical signals.

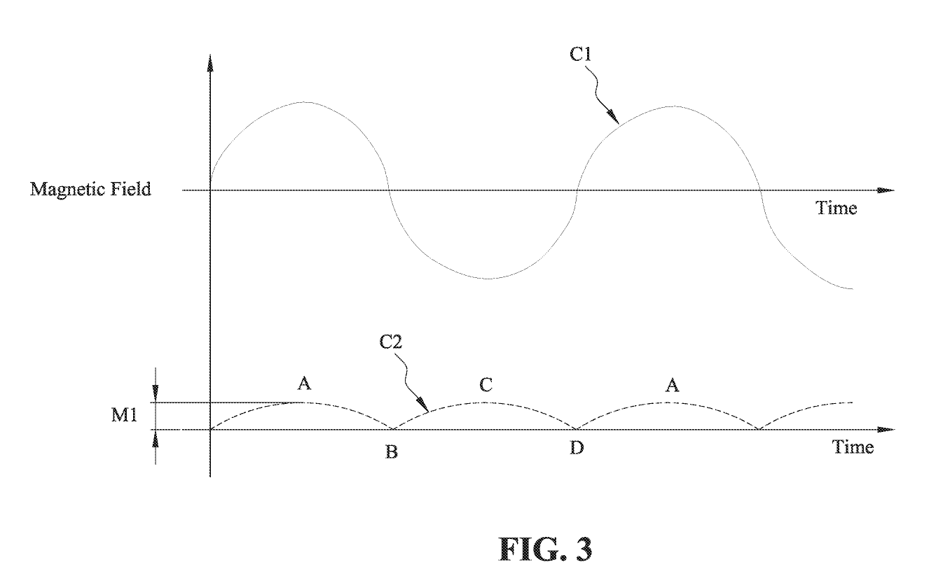

FIG. 3 is an oscillogram illustrating the electrical signals output by the light detection unit corresponding to the magnetic biosensor at different positions. As shown in FIGS. 2 and 3, when the magnetic biosensor 106 rotates to the position A or C, an area that the magnetic biosensor 106 shields the light rays passing through the liquid sample is d1, the light rays that passed through the sample cell 100 and enter the light detection unit 103 are more, the electrical signals output by the light detection unit 103 can be higher. When the magnetic biosensor 106 rotates to the position B or D, an area that the magnetic biosensor 106 shields the light rays passing through the liquid sample is d2 that is more than d1, and the electrical signals output by the light detection unit 103 are lower. An oscillogram of the electrical signals C2 output by the light detection unit 103 has a frequency that is two times of the electromagnetic frequency; moreover C1, due the oscillating magnetic field only drives the magnetic biosensor 106 to rotate, and a change of light rays caused by the rotation of the magnetic biosensor 106 is smaller, so that the change (amplitude) M1 of the oscillogram of the electrical signals C2 is smaller.

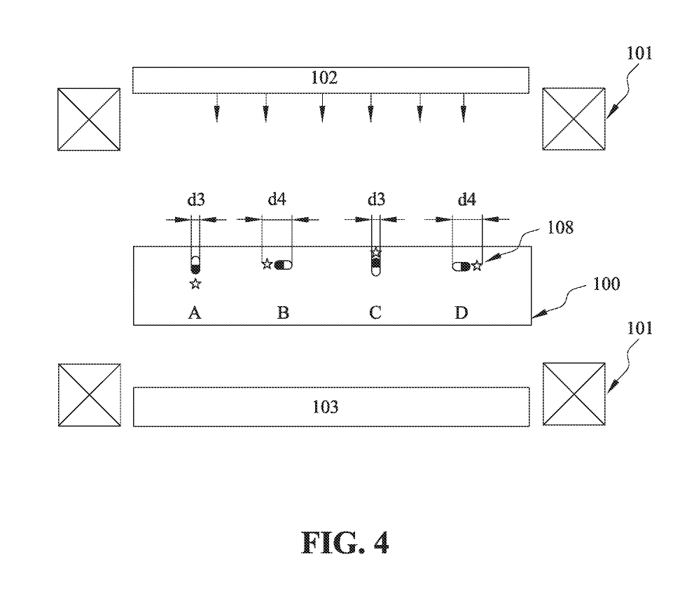

FIG. 4 is a side view schematically illustrating a state in the sample cell that the magnetic biosensor combines the detection object to form a magnetic cluster. As shown in FIG. 4, the magnetic biosensor 106 combines the detection object 107 to form a magnetic cluster 108 in the sample cell 100, the oscillating magnetic field generated by the electromagnetic generating unit 101 drives the magnetic biosensor 106 rotating along a same direction of positions A, B, C and D. Due a size of the magnetic cluster 108 is greater than a size of the magnetic biosensor 106, and a density of the magnetic cluster 108 is less than a density of uncombined the magnetic biosensor 106 and detection object 107, an effect of the rotation of the magnetic cluster 108 on the light rays is more obviously. When the magnetic cluster 108 rotates to the position A or C, an area that the magnetic cluster 108 shields the light rays passing through the liquid sample is d3 that is less than an area that the magnetic biosensor 106 and the detection object 107 shield the light rays, so that the electrical signals output by the light detection unit 103 is higher. When the magnetic cluster 108 rotates to the position B or D, an area that the magnetic cluster 108 shields the light rays passing through the liquid sample is d4 that is more than an area that the magnetic biosensor 106 and the detection object 107 shield the light rays (the magnetic biosensor 106 and the detection object 107 are partially overlapped on the passing path of the light rays), so that the electrical signals output by the light detection unit 103 is lower.

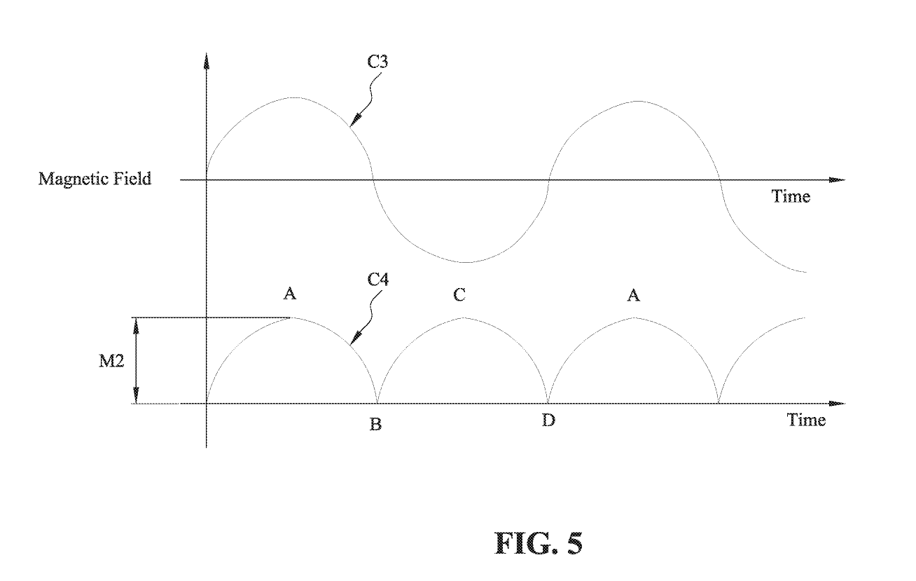

FIG. 5 is an oscillogram illustrating the electrical signals output by the light detection unit corresponding to the magnetic cluster at different positions. As shown in FIGS. 4 and 5, a frequency of the electrical signals C 4 output by the light detection unit 103 is two times of the magnetic field frequency C3; moreover, due the magnetic field drives the magnetic cluster 108 to rotate, and a change of light rays caused by the rotation of the magnetic cluster 108 is greater, so that the change (amplitude) M2 of the oscillogram of the electrical signals C4 output by the light detection unit 103 is greater.

Therefore, a result that the electrical signal change M2 is subtracted by the electrical change M1 (i.e. a difference between of the electrical signals based on the rotations of the magnetic cluster 108 and the magnetic biosensor 106) is proportional to an amount of the detection object 107 combining the magnetic biosensor 106, the amount of the objected to be detected 107 is represented as:

The amount of the detection object=(the electrical signal change M2−the electrical signal change M1)/k2, wherein k2 is a constant corresponding to specific magnetic biosensor 106 and the detection object 107 (EQ. 1).

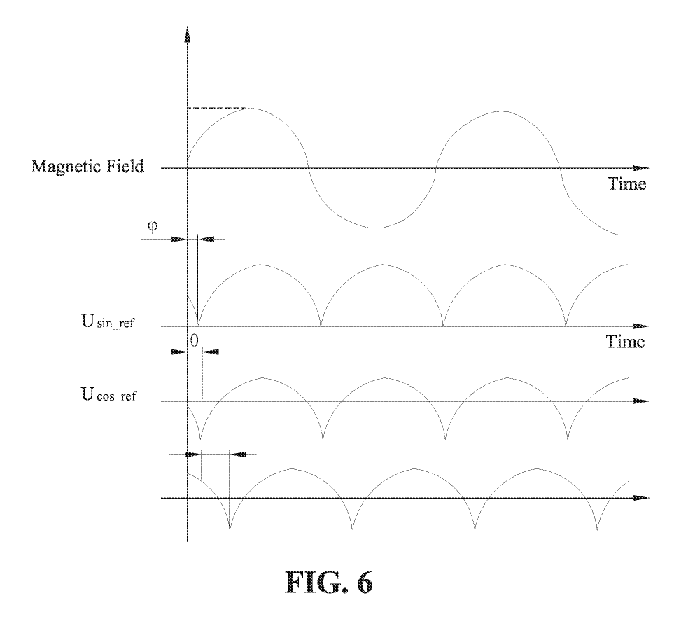

In order to detect the change of electrical signals output by the light detection unit 103, the lock-in amplifier 105 is used for calculating the change of the electrical signals. FIG. 6 is an oscillogram illustrating the electrical signals which are input the lock-in amplifier. The oscillating magnetic field m(t) of the electromagnetic generating unit 101, as shown in FIG. 6, is represented as EQ. 2, wherein M_{ac} represents a magnetic field intensity, and w represents magnetic field frequency.

m(t)=M_{ac}+sin(wt) EQ. 2

When the oscillating magnetic field drives the magnetic biosensor 106 rotating, the electrical signal Uᵢₙ output by the light detection unit 103 is not an oscillating signal of single frequency, but consisting of multiple signals of a frequency being two times of the magnetic field frequency. The electrical signals Uᵢₙ output by the light detection unit 103 is represented as EQ. 3, wherein φ represents a phase difference between the magnetic field m(t) and the electrical signals Uᵢₙ output by the light detection unit 103, b₀, b₂, b₄, b₆ represent amplitudes of the frequency.

Uᵢₙ=b₀+b₂*sin(2wt+φ)+b₄*sin(4wt+φ)+b₆*sin(6wt+φ)+ . . . , EQ. 3

In order to obtain the change of the electrical signals Uᵢₙ output by the light detection unit 103, a reference aggregate signal is set in the DSP 104. As shown in FIG. 6, the reference aggregate signal includes in-phase reference Uₛᵢₙ_{_}_{ref} and quadrature reference U_{cos}_{_}_{ref}, the references Uₛᵢₙ_{_}_{ref} and U_{cos}_{_}_{ref} also are consisting of multiple signals of a frequency being two times of the magnetic field frequency, direct current (DC) signal thereof must be zero, and a phase difference between the references Uₛᵢₙ_{_}_{ref} and U_{cos}_{_}_{ref} is 90°. The references Uₛᵢₙ_{_}_{ref} and U_{cos ref} are respectively represented as EQ. 4 and EQ. 5, wherein θ represent the phase difference between the oscillating magnetic field m(t) of the electromagnetic generating unit 101 and the reference Uₛᵢₙ_{_}_{ref}, u₀, u₂, u₄, u₆ represent amplitudes of the frequency. For frequencies that do not want to be detected, the amplitudes of the frequency are zero.

Uₛᵢₙ_{_}_{ref}=u₂*sin(2wt|θ)|u₄*sin(4wt|θ)|u₆*sin(6wt|θ)| . . . , EQ. 4

U_{cos ref}=u₂*cos(2wt+θ)+u₄*cos(4wt+θ)+u₆*cos(6wt+θ)+ . . . , EQ. 5

The DSP 104 transmits the references in-phase Uₛᵢₙ_{_}_{ref} and quadrature U_{cos}_{_}_{ref} to the lock-in amplifier 105; the lock-in amplifier 105 multiplies the electrical signals Uᵢₙ output by the light detection unit 103 with the reference Uₛᵢₙ_{_}_{ref}, and removes alternating current (AC) signal and retains the DC signal through integration; and an obtained DC signal is U_{In-Phase} (in-Phase component) represented as EQ. 6, wherein constant k is related to a time length of the integration.

$${U}_{I n - Phase} - {\sum }_{n = 1}^{∞} ( {U}_{i n} * {U}_{sin\_ref} ) = \frac{k}{2} cos ( ϕ - θ ) * [ {u}_{2} * {b}_{2} + {u}_{4} * {b}_{4} + {u}_{6} * {b}_{6} + … ] \qquad EQ . 6$$

Same as the calculation of U_{In-Phase}, the lock-in amplifier 105 multiplies the electrical signals Uᵢₙ output by the light detection unit 103 with the reference U_{cos ref}, and removes alternating current (AC) signal and retains the DC signal through integration; and an obtained DC signal is U_{quadrature} (quadrature component) represented as EQ. 7, wherein constant k is related to a time length of the integration.

$${U}_{quadrature} = {\sum }_{n = 1}^{∞} ( {U}_{i n} * {U}_{cos\_ref} ) - \frac{k}{2} * sin ( ϕ - θ ) * [ {u}_{2} * {b}_{2} + {u}_{4} * {b}_{4} + {u}_{6} * {b}_{6} + … ] \qquad EQ . 7$$

Due both in-Phase component of EQ. 6 and quadrature component of EQ. 7 are related to the phase θ and the phase φ, effects of the phase θ and the phase φ can be removed by using EQ. 8, the obtained result is only related to the constants of the aggregate reference signal and the change of the electrical signals of the light detection unit 103. When the selected constants u₂, u₄, u₆ of the aggregate reference signal are fixed, the obtained result is only related to the change M of the electrical signals Uᵢₙ output by the light detection unit 103; accordingly, the amount of the detection object 107 in the liquid sample can rapidly and accurately be obtained through calculation.

Change of the electrical signals

$$M = \sqrt{{U}_{i n - phase}^{2} \vert {U}_{quadrature}^{2}} = \begin{matrix}k \\ 2\end{matrix} * [ {u}_{2} * {b}_{2} \vert {u}_{4} * {b}_{4} \vert {u}_{6} * {b}_{6} \vert … ] \qquad EQ . 8$$

In an embodiment, the detection object is biotinylated anti-streptavidin, a biosensor having a combining uniqueness to the detection object is streptavidin. The biosensor streptavidin is a ˜60 kDa protein from Streptomycetes avidinii, contains four biotin-binding sites, and can be covalently coupled to the surface of specific magnetic nanoparticles. The detection process includes following steps: adding magnetic nanoparticles of a radius about 50 nm into a solution containing the streptavidin concentration of 500 ug/mL, allowing the streptavidin coating over the magnetic nanoparticles to form a magnetic biosensor solution; respectively mixing samples of 50 uL containing the biotinylated anti-streptavidin of concentrations in a range of 30-500 pM with the magnetic biosensor solution of 50 uL, disposing the mixed sample of 100 uL in the sample cell; reading a change of light rays of a laser (wavelength of 405 nm and intensity of 1 mw) passing through the mixed sample under a magnetic field (intensity of 2 mt and oscillating frequency of 20 Hz); then, driving movement of the magnetic biosensors in the mixed sample by using a stronger magnetic field (intensity of 80 mt), to allow the detection object biotinylated anti-streptavidin completely bonding to the streptavidin of the magnetic sensors, then, reading a change of light rays of the same laser passing through the mixed sample affected by the strong magnetic field; and, calculating the amounts of the detection objection biotinylated anti-streptavidin in each the mixed sample.

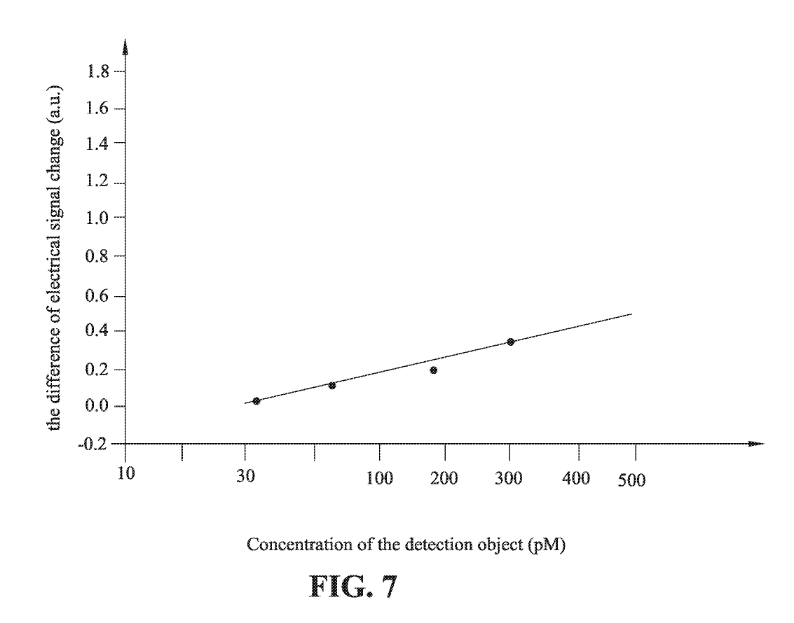

FIG. 7 is a scatter diagram illustrating the detection data of the electrical signal change to the detection objects of respective concentrations according to the aforesaid embodiment. As shown in FIG. 7, the vertical axis represents the differences (arbitrary unit) between the electric signal changes produced by the light rays passing through the detection object samples under the effect of different magnetic fields intensities, the horizontal axis represents the concentrations (pM) of the detection object, the line segment indicates that the differences of the electrical signal changes are proportional to the concentrations of the detection object, the slope of the line segment is k2 in EQ. 1. In the biomedical detection device of the present application, an interpolation equation (i.e. EQ. 1) for the detection objection concentration can be established by the use of the detection object sample of known concentrations, and the interpolation equation can be thus used for measuring the detection object of unknown concentrations.

In summary, in the biomedical detection device of the present application, the magnetic biosensor can combine the detection object to form the magnetic cluster, the electromagnetic generating unit can generate an oscillating magnetic field of single frequency to drive motions of the magnetic biosensor and the magnetic cluster, the light detection unit can output electrical signals based on a change of the light rays caused by motions of the magnetic biosensor and the magnetic cluster, when the light rays pass through the liquid sample, and the amount of the detection object in the liquid sample can rapidly and accurately be obtained through calculation of the change of the electrical signals output by the light detection unit.

Although the present application has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A biomedical detection device, comprising:
a sample cell, filled with a liquid sample containing a detection object and a magnetic biosensor capable of combining the detection object to form a magnetic cluster;
an electromagnetic generating unit, disposed at opposite two sides of the sample cell, and capable of generating an oscillating magnetic field of single frequency to drive motions of the magnetic biosensor and the magnetic cluster;
a light source, disposed at a side of the sample cell, and capable of emitting light rays to pass through the liquid sample; and
a light detection unit, disposed at another side of the sample cell oppositely to the light source, and capable of outputting electrical signals based on a change of the light rays caused by the motions of the magnetic biosensor and the magnetic cluster when the light rays pass through the liquid sample.

2. The biomedical detection device according to claim 1, a concentration of the magnetic biosensor in the liquid sample is 0.1-2000 μg/mL.

3. The biomedical detection device according to claim 1, wherein the single frequency of the oscillating magnetic field is 1-500 Hz, and an intensity of the oscillating magnetic field is 1-100 mT.

4. The biomedical detection device according to claim 1, wherein a wavelength of the light ray emitted by the light source is under 650 nm.

5. The biomedical detection device according to claim 3, wherein the light source comprises a polarized laser.

6. The biomedical detection device according to claim 1, further comprising a digital signal processor, for controlling the electromagnetic generating unit to generate the oscillating magnetic field of the single frequency, and storing a reference aggregate signal of a frequency being two times of the single frequency of the oscillating magnetic field.

7. The biomedical detection device according to claim 6, further comprising a lock-in amplifier, connecting to the light detection unit and the digital signal processor, and for measuring the change of the electrical signal based on the reference aggregate signal.

8. The biomedical detection device according to claim 6, wherein the reference aggregate signal comprises an in-phase component and a quadrature component.

9. The biomedical detection device according to claim 1, wherein an amount of the detection object is proportional to a difference of the intensity change between the light ray passing through the magnetic cluster and uncombined the detection object and the magnetic biosensor.
